(19) United States
(12) Patent Application Publication
(10) Pub. No.: US 2018/0214134 A1
(43) Pub. Date: Aug. 2, 2018

(54) ULTRASOUND DIAGNOSIS APPARATUS AND METHOD OF OPERATING THE SAME

(71) Applicant: SAMSUNG MEDISON CO., LTD., Hongcheon-gun (KR)

(72) Inventors: Beom-gyu KIM, Hongcheon-gun (KR); Gi-duck KIM, Hongcheon-gun (KR); Sun-mo YANG, Hongcheon-gun (KR); Eun-ho YANG, Hongcheon-gun (KR)

(73) Assignee: SAMSUNG MEDISON CO., LTD., Hongcheon-gun (KR)

(21) Appl. No.: 15/699,218

(22) Filed: Sep. 8, 2017

(30) Foreign Application Priority Data
Feb. 2, 2017 (KR) 10-2017-0014978

(51) Int. Cl. A61B 8/00; A61B 8/08

(52) U.S. Cl. CPC A61B 8/54; A61B 8/463; A61B 8/5276; A61B 8/488; A61B 8/469; A61B 8/481; A61B 8/4263

(57) ABSTRACT

An ultrasound diagnosis apparatus includes: a controller configured to obtain a real-time scan image based on ultrasound data about a region of interest, determine whether a movement of a probe occurs based on the ultrasound data, and when the movement of the probe does not occur for a first time or more, obtain an ultrasound still image in which at least one image quality parameter according to a preset standard is changed based on the ultrasound data; and a display configured to display the real-time scan image and the ultrasound still image.

### CROSS-REFERENCE TO RELATED APPLICATION

This application claims the benefit of Korean Patent Application No. 10-2017-0014978, filed on Feb. 2, 2017, in the Korean Intellectual Property Office, the disclosure of which is incorporated herein in its entirety by reference.

### BACKGROUND

### 1. Field

One or more embodiments relate to an ultrasound diagnosis apparatus and a method of operating the same, and more particularly, to an ultrasound diagnosis apparatus for obtaining an ultrasound still image having improved quality and a method of operating the ultrasound diagnosis apparatus.

### 2. Description of the Related Art

Ultrasound diagnosis apparatuses transmit ultrasound signals generated by transducers of a probe to an object and receive echo signals reflected from the object, thereby obtaining at least one image of an internal part of the object (e.g., soft tissue or blood flow). In particular, ultrasound diagnosis apparatuses are used for medical purposes including observation of the interior of an object, detection of foreign substances, and diagnosis of damage to the object. Such ultrasound diagnosis apparatuses provide high stability, display images in real time, and are safe due to the lack of radioactive exposure, compared to X-ray apparatuses. Therefore, ultrasound diagnosis apparatuses are widely used together with other image diagnosis apparatuses.

However, there may be a limitation in improving image quality to obtain an image displayed in real time by using an ultrasound diagnosis apparatus. For example, a user may adjust parameters for improving image quality including a gain of an ultrasound signal, a dynamic range, a scan line density, and a focus number, to obtain a real-time scan image. Although the user may improve image quality by adjusting the parameters, a frame rate according to real-time scanning has to be reduced due to a limit of an image processing speed of the ultrasound diagnosis apparatus.

When a real-time scan image, such as a cardiac ultrasound image in which a movement of an object is important, is to be obtained by an ultrasound diagnosis apparatus, a relatively high frame rate is required, and thus there is a limitation in improving image quality. Accordingly, sometimes, it may be difficult for a user of an ultrasound diagnosis apparatus to adjust parameters for improving image quality to obtain a real-time scan image.

### SUMMARY

One or more embodiments include obtaining an image having improved quality without reducing a frame rate of real-time scanning by automatically changing parameters for improving image quality at a time when a user is to obtain an ultrasound image.

One or more embodiments include improving user convenience by automatically changing parameters for improving image quality at a time when a user is to obtain an ultrasound image.

Additional aspects will be set forth in part in the description which follows and, in part, will be apparent from the description, or may be learned by practice of the presented embodiments.

According to one or more embodiments, an ultrasound diagnosis apparatus includes: a controller configured to obtain a real-time scan image based on ultrasound data about a region of interest, determine whether a movement of a probe occurs based on the ultrasound data, and when the movement of the probe does not occur for a first time or more, obtain an ultrasound still image in which at least one image quality parameter according to a preset standard is changed based on the ultrasound data; and a display configured to display the real-time scan image and the ultrasound still image.

After the first time elapses, the controller may be configured to obtain at least one ultrasound still image in which at least one image quality parameter is changed over time based on the ultrasound data.

After the first time elapses, the controller may be configured to obtain at least one ultrasound still image in which at least one image quality parameter preset by a user is changed based on the ultrasound data.

The controller may be configured to determine whether a movement of an object included in the region of interest exceeds a predetermined amount during a second time based on the ultrasound data, wherein when the movement of the object exceeds the predetermined amount, the controller is configured to additionally obtain the ultrasound data about the region of interest.

The ultrasound diagnosis apparatus may further include a user input receiver configured to receive a user input, wherein the controller is configured to receive the user input for storing the ultrasound still image and store the ultrasound still image corresponding to the user input.

The controller may be configured to store the ultrasound still image to which an image quality parameter changed in a time interval corresponding to a time when the user input is received is applied.

The controller may be configured to store the ultrasound still image obtained before the time interval.

The display may be configured to display the ultrasound still image so that the ultrasound still image overlaps an outer portion of the real-time scan image.

The display may be configured to sequentially display a plurality of the ultrasound still images to which image quality parameters that are changed are applied in an order in which the image quality parameters are applied.

The real-time scan image may include a brightness (B) mode, a color (C) mode, a continuous wave Doppler (CW) mode, or a contrast enhanced image.

According to one or more embodiments, an ultrasound diagnosis method includes: obtaining a real-time scan image based on ultrasound data about a region of interest; determining whether a movement of a probe occurs based on the ultrasound data; when the movement of the probe does not occur for a first time or more, obtaining an ultrasound still image in which at least one image quality parameter according to a preset standard is changed based on the ultrasound data; and displaying the real-time scan image and the ultrasound still image.

### BRIEF DESCRIPTION OF THE DRAWINGS

These and/or other aspects will become apparent and more readily appreciated from the following description of the embodiments, taken in conjunction with the accompanying drawings in which reference numerals denote structural elements and:

FIG. 1 is a block diagram illustrating a configuration of an ultrasound diagnosis apparatus according to an embodiment;

FIGS. 2A, 2B, and 2C are views illustrating ultrasound diagnosis apparatuses according to an embodiment;

FIG. 3 is a block diagram illustrating a configuration of an ultrasound diagnosis apparatus according to an embodiment;

FIG. 4A is a real-time scan image and an ultrasound still image displayed by the ultrasound diagnosis apparatus according to an embodiment;

FIG. 4B is an ultrasound still image displayed by the ultrasound diagnosis apparatus over time according to an embodiment;

FIG. 4C is a view illustrating a real-time scan image and an ultrasound still image displayed by the ultrasound diagnosis apparatus according to an embodiment;

FIG. 5 is a view for explaining an operation performed by the ultrasound diagnosis apparatus to display an ultrasound still image according to a user's setting value according to an embodiment;

FIG. 6A is a real-time scan image and an ultrasound still image displayed by the ultrasound diagnosis apparatus according to an embodiment;

FIG. 6B is an ultrasound still image displayed by the ultrasound diagnosis apparatus over time according to an embodiment;

FIG. 7A is a real-time scan image and an ultrasound still image displayed by the ultrasound diagnosis apparatus according to an embodiment;

FIG. 7B is an ultrasound still image obtained by the ultrasound diagnosis apparatus according to an embodiment; and

FIG. 8 is a flowchart of a method of operating the ultrasound diagnosis apparatus, according to an embodiment.

### DETAILED DESCRIPTION

Embodiments of the present disclosure will now be described in detail. However, the present disclosure is not limited to the embodiments disclosed below, but can be implemented in various forms. The following embodiments are described in order to enable one of ordinary skill in the art to embody and practice the invention.

Throughout the drawings, the same reference numerals denote the same elements. The present specification does not describe all elements of embodiments, and general content in the technical field to which the present disclosure pertains or repeated content between embodiments will not be explained. The term ‘part’ or ‘portion’ used herein may be implemented as software or hardware, and according to embodiments, a plurality of ‘parts’ or ‘portions’ may be implemented as one unit or element or one ‘part’ or ‘portion may include a plurality of units or elements. The operation principle and embodiments of the present disclosure will now be explained with reference to the attached drawings.

In the preset specification, an image may include any medical image acquired by various medical imaging apparatuses such as a magnetic resonance imaging (MRI) apparatus, a computed tomography (CT) apparatus, an ultrasound imaging apparatus, or an X-ray apparatus.

Also, in the present specification, an ‘object’, which is a thing to be imaged, may include a human, an animal, or a part thereof. For example, an object may include a part of a human, that is, an organ or a tissue, or a phantom.

Throughout the specification, an “ultrasound image” refers to an image of an object processed based on ultrasound signals transmitted to the object and reflected therefrom.

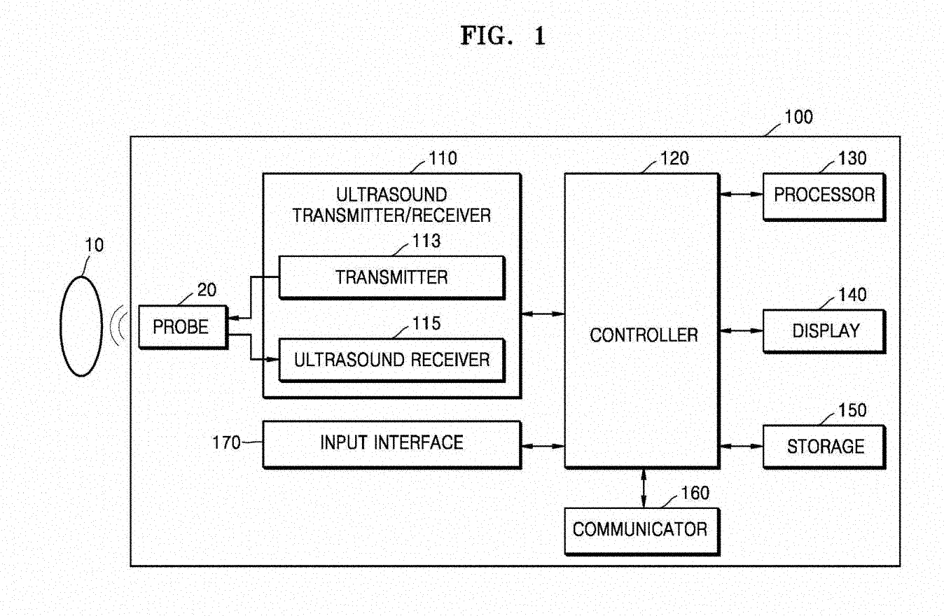

FIG. 1 is a block diagram illustrating a configuration of an ultrasound diagnosis apparatus 100 according to an embodiment.

Referring to FIG. 1, the ultrasound diagnosis apparatus 100 may include a probe 20, an ultrasound transceiver 110, a controller 120, an image processor 130, one or more displays 140, a storage 150, a communicator 160, and an input interface 170.

The ultrasound diagnosis apparatus 100 may be a cart-type or a portable-type ultrasound diagnosis apparatus. Examples of the portable-type ultrasound diagnosis apparatus 100 may include a smart phone, a laptop computer, a personal digital assistant (PDA), and a tablet personal computer (PC), each of which may include a probe and a software application, but embodiments are not limited thereto.

The probe 20 may include a plurality of transducers. The plurality of transducers may transmit ultrasound signals to an object 10 in response to transmission signals received by the probe 20, from a transmitter 113. The plurality of transducers may receive ultrasound signals reflected from the object 10 to generate reception signals. In addition, the probe 20 and the ultrasound diagnosis apparatus 100 may be formed in one body (e.g., disposed in a single housing), or the probe 20 and the ultrasound diagnosis apparatus 100 may be formed separately (e.g., disposed separately in separate housings) but linked wirelessly or via wires. In addition, the ultrasound diagnosis apparatus 100 may include one or more probes 20 according to embodiments.

The controller 120 may control the transmitter 113 to generate transmission signals to be applied to each of the plurality of transducers based on a position and a focal point of the plurality of transducers included in the probe 20.

The controller 120 may control an ultrasound receiver 115 to generate ultrasound data by converting reception signals received from the probe 20 from analog to digital signals and summing the reception signals converted into digital form, based on a position and a focal point of the plurality of transducers.

The image processor 130 may generate an ultrasound image by using ultrasound data generated from the ultrasound receiver 115.

The display 140 may display a generated ultrasound image and various pieces of information processed by the ultrasound diagnosis apparatus 100. The ultrasound diagnosis apparatus 100 may include one or more displays 140 according to the present embodiment. The display 140 may include a touch screen in combination with a touch panel.

The controller 120 may control the operations of the ultrasound diagnosis apparatus 100 and flow of signals between the internal elements of the ultrasound diagnosis apparatus 100. The controller 120 may include a memory for storing a program or data to perform functions of the ultrasound diagnosis apparatus 100 and a processor for processing the program or data. Also, the controller 120 may control the operation of the ultrasound diagnosis apparatus 100 by receiving a control signal from the input interface 170 or an external apparatus.

The ultrasound diagnosis apparatus 100 may include the communicator 160 and may be connected to external apparatuses, for example, servers, medical apparatuses, and portable devices such as smart phones, tablet personal computers (PCs), wearable devices, etc., via the communicator 160.

The communicator 160 may include at least one element capable of communicating with the external apparatuses. For example, the communicator 160 may include at least one among a short-range communication module, a wired communication module, and a wireless communication module.

The communicator 160 may receive a control signal and data from an external apparatus and transmit the received control signal to the controller 120 so that the controller 120 may control the ultrasound diagnosis apparatus 100 in response to the received control signal.

Alternatively, the controller 120 may transmit a control signal to the external apparatus via the communicator 160 so that the external apparatus may be controlled in response to the control signal of the controller 120.

For example, the external apparatus connected to the ultrasound diagnosis apparatus 100 may process the data of the external apparatus in response to the control signal of the controller 120 received via the communicator 160.

A program for controlling the ultrasound diagnosis apparatus 100 may be installed in the external apparatus. The program may include command languages to perform part of operation of the controller 120 or the entire operation of the controller 120.

The program may be pre-installed in the external apparatus or may be installed by a user of the external apparatus by downloading the program from a server that provides applications. The server that provides applications may include a recording medium where the program is stored.

The storage 150 may store various data or programs for driving and controlling the ultrasound diagnosis apparatus 100, input and/or output ultrasound data, ultrasound images, etc.

The input interface 170 may receive a user's input to control the ultrasound diagnosis apparatus 100 and may include a keyboard, button, keypad, mouse, trackball, jog switch, knob, a touchpad, a touch screen, a microphone, a motion input means, or a biometrics input means (e.g., iris recognition or fingerprint recognition), but embodiments are not limited thereto.

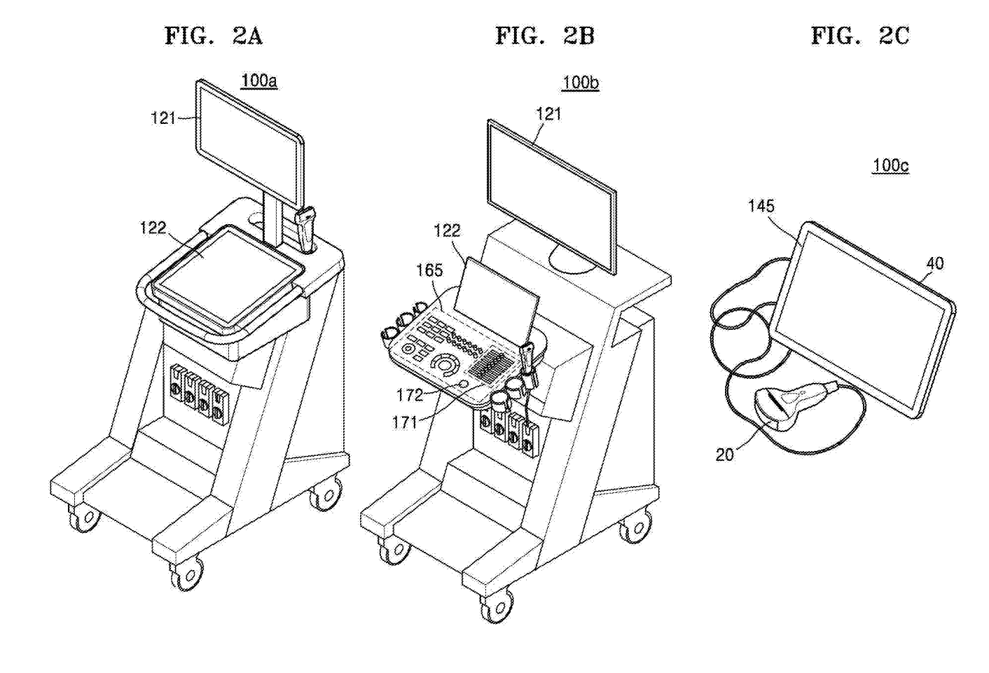

An example of the ultrasound diagnosis apparatus 100 according to an embodiment is described below with reference to FIGS. 2A, 2B, and 2C.

FIGS. 2A, 2B, and 2C are views illustrating ultrasound diagnosis apparatuses according to an embodiment.

Referring to FIGS. 2A and 2B, an ultrasound diagnosis apparatus 100a or 100b may include a main display 121 and a sub-display 122. At least one among the main display 121 and the sub-display 122 may include a touch screen. The main display 121 and the sub-display 122 may display ultrasound images and/or various information processed by the ultrasound diagnosis apparatus 100a or 100b. The main display 121 and the sub-display 122 may provide graphical user interfaces (GUI), thereby receiving a user's inputs of data to control the ultrasound diagnosis apparatus 100a or 100b. For example, the main display 121 may display an ultrasound image and the sub-display 122 may display a control panel to control display of the ultrasound image as a GUI. The sub-display 122 may receive an input of data to control the display of an image through the control panel displayed as a GUI. The ultrasound diagnosis apparatus 100a or 100b may control the display of the ultrasound image on the main display 121 by using the input control data.

Referring to FIG. 2B, the ultrasound diagnosis apparatus 100b may include a control panel 165. The control panel 165 may include buttons, trackballs, jog switches, or knobs, and may receive data to control the ultrasound diagnosis apparatus 100b from the user. For example, the control panel 165 may include a time gain compensation (TGC) button 171 and a freeze button 172. The TGC button 171 is to set a TGC value for each depth of an ultrasound image. Also, when an input of the freeze button 172 is detected during scanning an ultrasound image, the ultrasound diagnosis apparatus 100b may keep displaying a frame image at that time point.

The buttons, trackballs, jog switches, and knobs included in the control panel 165 may be provided as a GUI to the main display 121 or the sub-display 122.

Referring to FIG. 2C, an ultrasound diagnosis apparatus 100c may be portable. An example of the portable ultrasound diagnosis apparatus 100c may include, for example, smart phones including probes and applications, laptop computers, personal digital assistants (PDAs), or tablet PCs, but embodiments are not limited thereto.

The ultrasound diagnosis apparatus 100c may include the probe 20 and a main body 40. The probe 20 may be connected to one side of the main body 40 by wire or wirelessly. The main body 40 may include a touch screen 145. The touch screen 145 may display an ultrasound image, various pieces of information processed by the ultrasound diagnosis apparatus 100c, and a GUI.

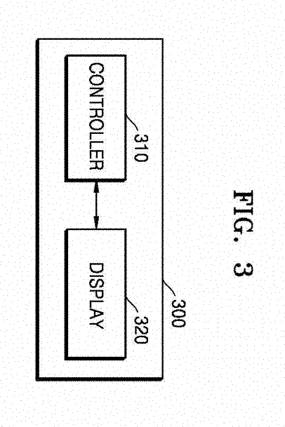

FIG. 3 is a block diagram illustrating a configuration of an ultrasound diagnosis apparatus 300 according to an embodiment.

Referring to FIG. 3, the ultrasound diagnosis apparatus 300 may include a controller 310 and a display 320. The controller 310 may be included in the controller 120 or the image processor 130 of FIG. 1, and the display 320 may be included in the display 140 of FIG. 1.

The controller 310 according to an embodiment obtains a real-time scan image based on ultrasound data about a region of interest.

Also, the controller 310 may determine whether a movement of a probe (not shown) occurs based on the ultrasound data. The controller 310 may determine whether the movement of the probe occurs by using a sensor included in the probe.

When the movement of the probe does not occur for a first time or more, the controller 310 obtains an ultrasound still image in which at least one image quality parameter according to a preset standard for the region of interest is changed.

The display 320 displays the real-time scan image and the ultrasound still image.

The real-time scan image may be any of various images. For example, the real-time scan image may be at least one from among an amplitude (A) mode image, a brightness (B) mode image, a color (C) mode image, a Doppler (D) mode image, a continuous wave Doppler (CW) mode image, and a contrast enhanced ultrasound (CEUS) image. Also, according to an embodiment, the real-time scan image may be a two-dimensional (2D) or three-dimensional (3D) image.

The ultrasound still image may be a still image obtained after the movement of the probe stops. For example, the ultrasound still image may be an ultrasound still image in which at least one image quality parameter is changed when compared with the real-time scan image including the region of interest. The ultrasound still image may be obtained based on the ultrasound data about the region of interest corresponding to a time when the movement of the probe stops. The region of interest refers to a region shown on the real-time scan image displayed at a time when the movement of the probe stops.

According to an embodiment, a user may stop the movement of the probe at a time when the region of interest including an object is shown well on the real-time scan image obtained by the ultrasound diagnosis apparatus 300.

A case where a first time elapses after the movement of the probe stops will now be explained. The first time may be a value preset by the ultrasound diagnosis apparatus 300 or may be set by the user.

After the first time elapses, the controller 310 may obtain at least one ultrasound still image in which at least one image quality parameter is changed over time based on the ultrasound data.

Also, after the first time elapses, the controller 310 may obtain at least one ultrasound still image in which at least one image quality parameter preset by the user is changed based on the ultrasound data.

The image quality parameter according to an embodiment may include an ultrasound signal transmission/reception parameter and a post-processing parameter.

The post-processing parameter may include an image sharpness, a spatial compounding image (SCI) index, an imaging filter index, and a spatial filter index.

Also, when the real-time scan image and the ultrasound still image are color images, the image quality parameter may include an ensemble value.

Also, the controller 310 may determine whether a movement of the object included in the region of interest exceeds a predetermined amount during a second time based on the ultrasound data.

The second time may refer to a time taken to obtain the ultrasound data in order to obtain a contrast enhanced image.

Also, examples of a case where the movement of the object exceeds the predetermined amount includes a case where blur based on the movement of the object in the ultrasound still image is observed with the eyes.

When the movement of the object during the second time exceeds the predetermined amount, the controller 310 may additionally obtain the ultrasound data about the region of interest. For example, when it is determined that the movement of the object during the second time exceeds the predetermined amount, blur occurs in an ultrasound image due to the movement of the object. Accordingly, the ultrasound diagnosis apparatus 300 obtains again the ultrasound data during the second time in order to improve image quality.

The ultrasound diagnosis apparatus 300 may further include a user input receiver (not shown) for receiving a user input. For example, the user input receiver may include a freeze button.

The ultrasound diagnosis apparatus 300 may receive a freeze button input while scanning an ultrasound image. When the ultrasound diagnosis apparatus 300 receives the freeze button input, the ultrasound diagnosis apparatus 300 may maintain a state where a frame image at a time when the freeze button input is received is displayed.

The ultrasound diagnosis apparatus 300 may further include a storage (not shown) for storing the ultrasound still image. The controller 310 may be configured to store the ultrasound still image corresponding to a user input.

According to an embodiment, the controller 310 may be configured to store the ultrasound still image to which the image quality parameter changed in a time interval corresponding to a time when the user input is received is applied.

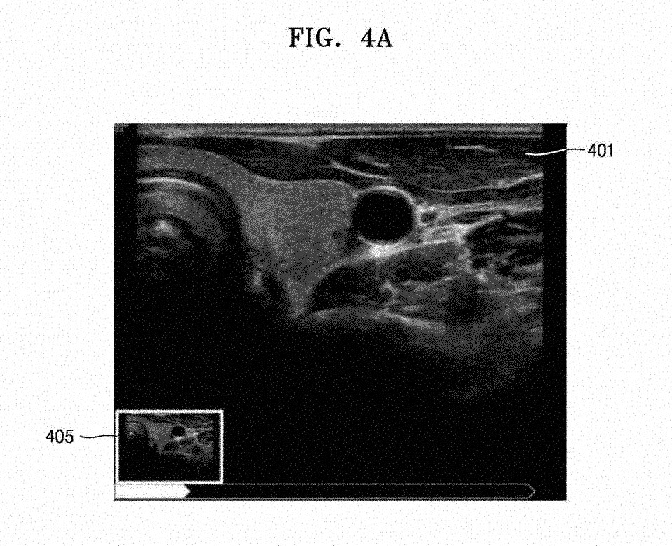

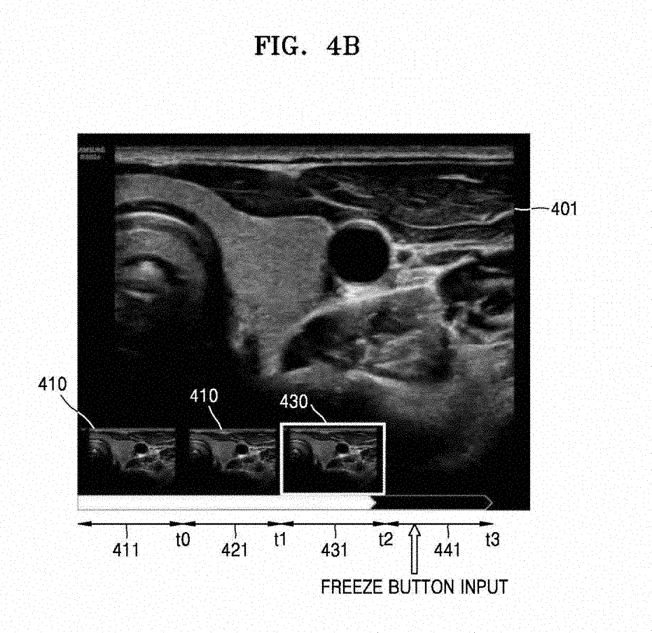

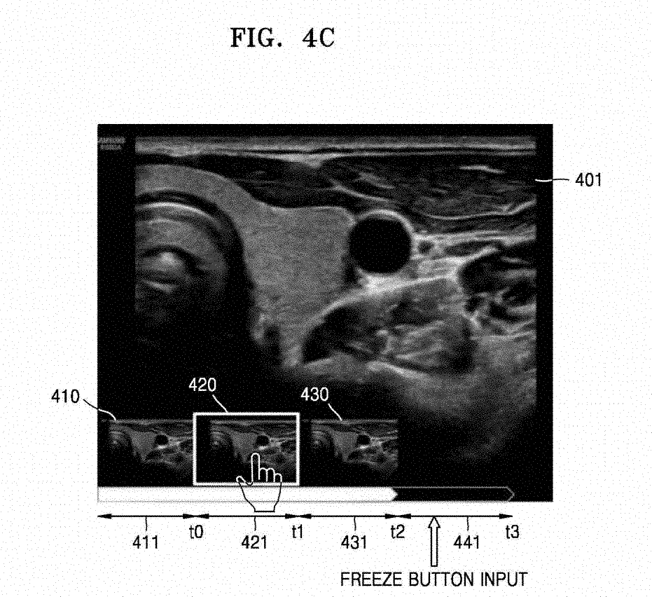

FIG. 4A is a real-time scan image and an ultrasound still image displayed by the ultrasound diagnosis apparatus 300 according to an embodiment.

The ultrasound diagnosis apparatus 300 may display a real-time scan image 401 obtained based on ultrasound data about a region of interest.

Also, the ultrasound diagnosis apparatus 300 may display an ultrasound still image 405 so that the ultrasound still image 405 overlaps an outer portion of the real-time scan image 401.

Also, referring to FIG. 4A, the ultrasound diagnosis apparatus 300 may display the ultrasound still image 405 on a lower end of the real-time scan image 401.

The ultrasound still image 405 may be an image obtained when a movement of a probe does not occur for a first time or more.

The ultrasound diagnosis apparatus 300 may determine whether the movement of the probe does not occur based on a movement of an image obtained from the ultrasound data. In detail, when the movement of the image is within a predetermined range of values, the ultrasound diagnosis apparatus 100 may determine that the movement of the probe does not occur. For example, when a user stops the probe in order to more closely look at the region of interest, the movement of the probe may not occur.

The ultrasound still image 405 may be obtained after the first time during which the movement of the probe does not occur elapses. Alternatively, the ultrasound still image 405 may be obtained even before the first time elapses. In this case, the ultrasound diagnosis apparatus 300 may display the ultrasound still image 405 after the first time during which the movement of the probe does not occur elapses.

The ultrasound still image 405 may be an image in which at least one image quality parameter according to a preset standard is changed when compared with the real-time scan image 401.

For example, the scan line density of the ultrasound still image 405 may be is greater than that of the real-time scan image 401. Also, when the ultrasound still image 405 is a multi-focus image, the real-time scan image 401 may be a single-focus image.

Even before the user presses the freeze button, the ultrasound diagnosis apparatus 300 may obtain the ultrasound still image 405 in which at least one image quality parameter is changed based on the event that the user stops the movement of the probe during the first time. The image quality of the ultrasound still image 405 in which at least one image quality parameter is changed may be better than that of the real-time scan image 401.

FIG. 4B is an ultrasound still image displayed by the ultrasound diagnosis apparatus 300 over time according to an embodiment.

After a first time elapses, the ultrasound diagnosis apparatus 300 may obtain one or more ultrasound still images, e.g., first through third ultrasound still images 410, 420, and 430, in which at least one image quality parameter is changed over time based on ultrasound data.

The first ultrasound still image 410 may be an image in which a first image quality parameter is changed when compared with the real-time scan image 401.

The second ultrasound still image 420 may be an image in which a second image quality parameter is changed when compared with the real-time scan image 401.

The third ultrasound still image 430 may be an image in which a third image quality parameter is changed when compared with the real-time scan image 401.

According to an embodiment, image processing of the second ultrasound still image 420 may be longer than the first ultrasound still image 410. Also, image processing of the third ultrasound still image 430 be longer than the second ultrasound still image 420. In this case, the quality of the third ultrasound still image 430 may be image quality most preferred by a user.

Each of the first image quality parameter, the second image quality parameter, and the third image quality parameter may include one or more parameters.

For example, the first image quality parameter may include a scan line density and the number of focuses. The second image quality parameter may include a scan line density, the number of focuses, an image sharpness, and an SCI index. The third image quality parameter may include a scan line density, the number of focuses, an image sharpness, an SCI index, an imaging filter index, and a spatial filter index.

For example, the second image quality parameter may include the first image quality parameter, and the third image quality parameter may include the second image quality parameter.

The first image quality parameter, the second image quality parameter, and the third image quality parameter are exemplary, and various image quality parameters may be included.

The first ultrasound still image 410 may be an image obtained in a first time interval 411. The ultrasound diagnosis apparatus 300 may display the first ultrasound still image 410 from a point of time to.

The second ultrasound still image 420 may be an image obtained in a second time interval 421. The ultrasound diagnosis apparatus 300 may display the second ultrasound still image 420 from a point of time t1 after the point of time t0.

The third ultrasound still image 430 may be an image obtained in a third time interval 431. The ultrasound diagnosis apparatus 300 may display the third ultrasound still image 430 from a point of time t2 after the point of time t1.

According to an embodiment, quality of the second ultrasound still image 420 may be better than that of the first ultrasound still image 410. Also, quality of the third ultrasound still image 430 may be better than that of the second ultrasound still image 420.

The ultrasound diagnosis apparatus 300 according to an embodiment may enlarge and display the first through third ultrasound still images 410, 420, and 430 in order for the user to check the quality of the first through third ultrasound still images 410, 420, and 430. The user may provide a freeze input when it is determined that an image having desired quality is obtained by the ultrasound diagnosis apparatus 300.

The ultrasound diagnosis apparatus 300 may receive a freeze input from the user in a fourth time interval 441 after the point of time t2. The ultrasound diagnosis apparatus 300 may store the third ultrasound still image 430 corresponding to the point of time t2.

FIG. 4C is a real-time scan image and an ultrasound still image displayed by the ultrasound diagnosis apparatus 300 according to an embodiment.

The first ultrasound still image 410, the second ultrasound still image 420, and the third ultrasound still image 430 of FIG. 4C may respectively correspond to those of FIG. 4B.

Referring to FIG. 4C, the ultrasound diagnosis apparatus 300 may receive a freeze input from the user in the fourth time interval 441.

The user of the ultrasound diagnosis apparatus 300 may compare the first ultrasound still image 410, the second ultrasound still image 420, and the third ultrasound still image 430, and may prefer the quality of the second ultrasound still image 420. In this case, the user may provide an input for storing the second ultrasound still image 420 to the ultrasound diagnosis apparatus 300.

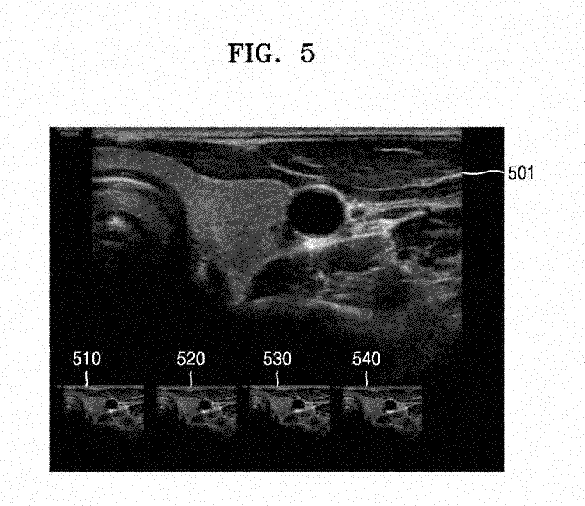

FIG. 5 is a view for explaining an operation performed by the ultrasound diagnosis apparatus 300 to display an ultrasound still image according to a user's setting value according to an embodiment.

The ultrasound diagnosis apparatus 300 may obtain at least one ultrasound still image when a movement of a probe does not occur for a first time or more.

A first ultrasound still image 510, a second ultrasound still image 520, a third ultrasound still image 530, and a fourth ultrasound still image 540 of FIG. 5 may be images to which different image quality parameters are applied.

The image quality parameters that are changed and applied to the first ultrasound still image 510, the second ultrasound still image 520, the third ultrasound still image 530, and the fourth ultrasound still image 540 may be parameters preset by the user.

The user may store at least one from among the first ultrasound still image 510, the second ultrasound still image 520, the third ultrasound still image 530, and the fourth ultrasound still image 540.

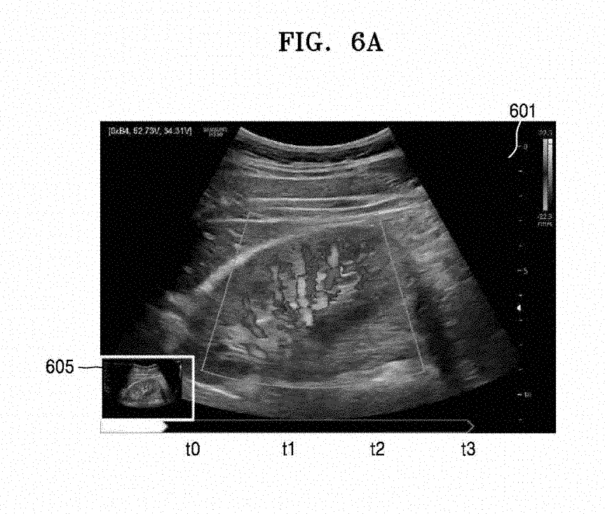

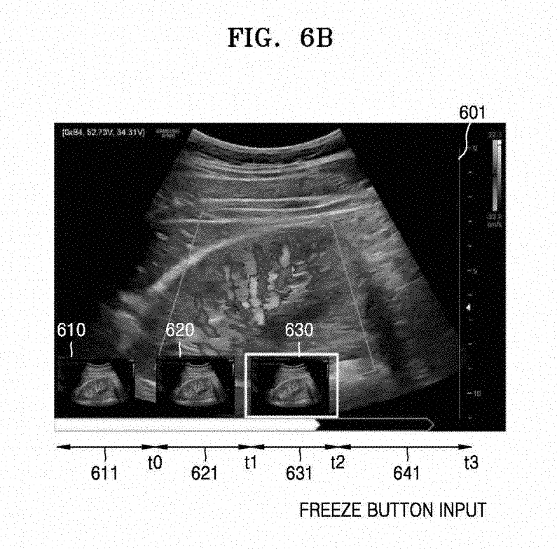

FIG. 6A is a real-time scan image and an ultrasound still image displayed by the ultrasound diagnosis apparatus 300 according to an embodiment.

The ultrasound diagnosis apparatus 300 may display a real-time scan image 601 and an ultrasound still image 605 obtained based on ultrasound data about a region of interest.

The real-time scan image 601 may be one from among, for example, an A mode image, a B mode image, a C mode image, a D mode image, a CW mode image, and a CEUS image. Also, the real-time scan image 601 may be a 2D image or a 3D image.

An image quality parameter that is changed in the ultrasound still image 605 may vary according to an image mode of the real-time scan image 601.

For example, when the real-time scan image 601 is a C mode image, as shown in FIG. 6A, the ultrasound diagnosis apparatus 300 may obtain the ultrasound still image 605 in which an ensemble value is improved when compared with the real-time scan image 601.

Also, an image quality parameter that is changed in the ultrasound still image 605 may vary according to whether the real-time scan image 601 is a 2D image or a 3D image.

FIG. 6B is an ultrasound still image displayed by the ultrasound diagnosis apparatus 300 over time according to an embodiment.

A first ultrasound still image 610 may be an image obtained in a first time interval 611. The ultrasound diagnosis apparatus 300 may display the first ultrasound still image 610 from a point of time to.

The second ultrasound still image 620 may be an image obtained in a second time interval 621. The ultrasound diagnosis apparatus 300 may display the second ultrasound still image 620 from a point of time t1 after the point of time t0.

The third ultrasound still image 630 may be an image obtained in a third time interval 631. The ultrasound diagnosis apparatus 300 may display the third ultrasound still image 630 from a point of time t2 after the point of time t1.

The first ultrasound still image 610 may be an image in which a first image quality parameter is changed when compared with the real-time scan image 601. When the real-time scan image 601 is a C mode image, the first image quality parameter may include an ensemble value.

The second ultrasound still image 620 may be an image in which a second image quality parameter is changed when compared with the real-time scan image 601. When the real-time scan image 601 is a C mode image, the second image quality parameter may include an ensemble value and a scan line density.

The third ultrasound still image 630 may be an image in which a third image quality parameter is changed when compared with the real-time scan image 601. When the real-time scan image 601 is a C mode image, the third image quality parameter may include an ensemble value, a scan line density, and a spatial density index.

The first image quality parameter, the second image quality parameter, and the third image quality parameter are exemplary, and various image quality parameters may be included.

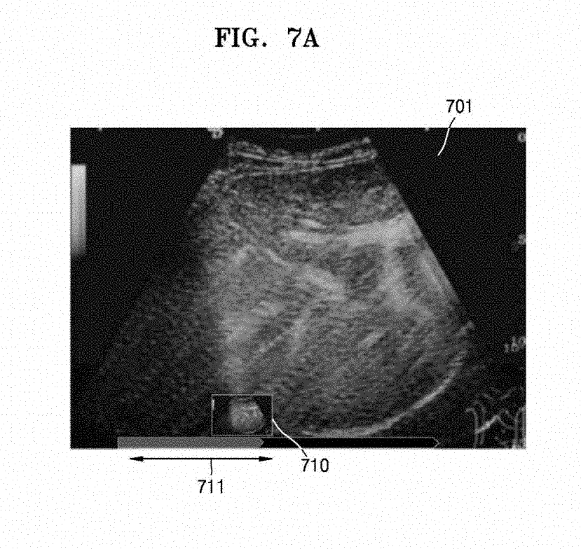

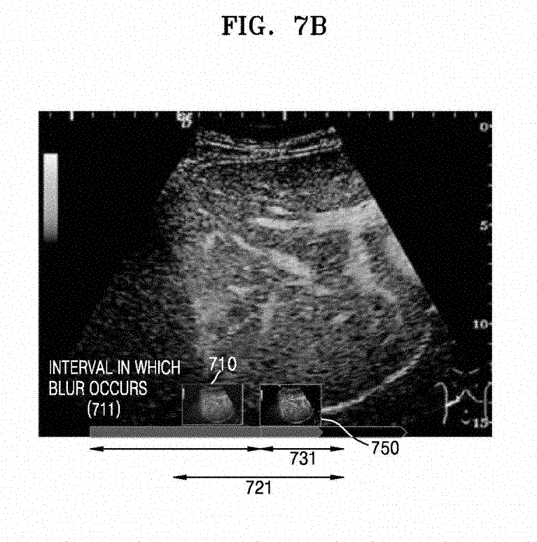

FIG. 7A is a real-time scan image and an ultrasound still image displayed by the ultrasound diagnosis apparatus 300 according to an embodiment.

Referring to FIG. 7A, a real-time scan image 701 may be a CEUS image.

The ultrasound diagnosis apparatus 300 may obtain an ultrasound still image 710 when a movement of a probe does not occur for a first time or more.

When the real-time scan image 701 is a CEUS image, the ultrasound diagnosis apparatus 300 may spend a second time 711 to obtain the ultrasound still image 710.

The ultrasound diagnosis apparatus 300 may obtain the ultrasound still image 710 based on ultrasound data obtained during the second time 711.

When a movement of an object included in a region of interest occurs during the second time 711, the ultrasound diagnosis apparatus 300 may obtain the ultrasound still image 710 in which blur occurs.

FIG. 7B is an ultrasound still image obtained by the ultrasound diagnosis apparatus 300 according to an embodiment.

The ultrasound diagnosis apparatus 300 may determine whether a movement of an object included in a region of interest during the second time 711 exceeds a predetermined amount based on ultrasound data.

Referring to FIG. 7B, when the movement of the object exceeds the predetermined amount, blur may be observed with the eyes in the ultrasound still image 710 obtained based on the ultrasound data during the second time 711.

When the movement of the object exceeds the predetermined amount during the second time 711, the ultrasound diagnosis apparatus 300 may additionally obtain the ultrasound data about the region of interest. The ultrasound diagnosis apparatus 30 may additionally obtain the ultrasound data about the region of interest during an additional acquisition time 731.

The ultrasound diagnosis apparatus 300 may obtain a new ultrasound still image 750 based on ultrasound data obtained during a third time 721.

When the movement of the object included in the region of interest during the third time 721 does not exceed the predetermined amount, the sharpness of the new ultrasound still image 750 may be better than that of the ultrasound still image 710.

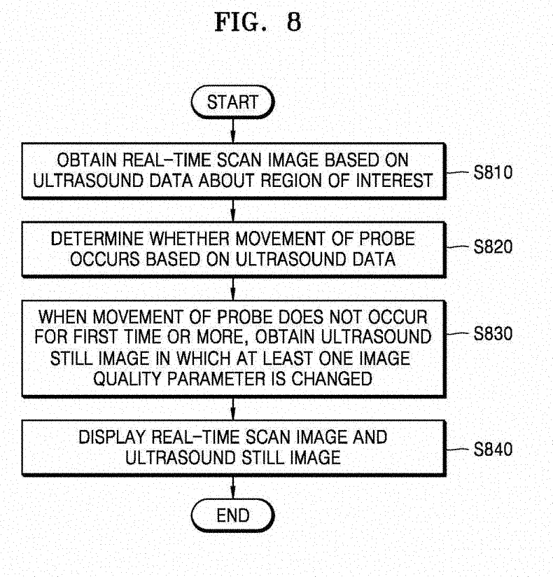

FIG. 8 is a flowchart of a method of operating the ultrasound diagnosis apparatus 300, according to an embodiment.

In operation S810, the ultrasound diagnosis apparatus 300 may obtain a real-time scan image based on ultrasound data about a region of interest.

In operation S820, the ultrasound diagnosis apparatus 300 may determine whether a movement of a probe occurs based on the ultrasound data.

In operation S830, when the movement of the probe does not occur for a first time or more, the ultrasound diagnosis apparatus 300 may obtain an ultrasound still image in which at least one image quality parameter according to a preset standard is changed based on the ultrasound data.

In operation S840, the ultrasound diagnosis apparatus 300 may display the real-time scan image and the ultrasound still image.

According to the one or more embodiments, since parameters for improving image quality are automatically changed at a time when a user is to obtain an ultrasound image, an image having improved quality may be obtained without reducing a frame rate of real-time scanning.

According to the one or more embodiments, since parameters for improving image quality are automatically changed at a time when a user is to obtain an ultrasound image, user convenience may be improved.

The present disclosure may be embodied as computer-readable recording media for storing computer-executable instructions and data. The instructions may be stored as program code, and when the instructions are executed by a processor, a predetermined program module may be generated to perform a predetermined operation. Also, when the instructions are executed by the processor, predetermined operations of the one or more embodiments may be performed.

What is claimed is:

1. An ultrasound diagnosis apparatus comprising:
a controller configured to
obtain a real-time scan image based on ultrasound data about a region of interest,
determine whether a movement of a probe occurs based on the ultrasound data, and
when the movement of the probe does not occur for a first time or more, obtain an ultrasound still image in which at least one image quality parameter according to a preset standard is changed based on the ultrasound data; and
a display configured to display the real-time scan image and the ultrasound still image.

2. The ultrasound diagnosis apparatus of claim 1, wherein, after the first time elapses, the controller is configured to obtain at least one ultrasound still image in which at least one image quality parameter is changed over time based on the ultrasound data.

3. The ultrasound diagnosis apparatus of claim 1, wherein, after the first time elapses, the controller is configured to obtain at least one ultrasound still image in which at least one image quality parameter preset by a user is changed based on the ultrasound data.

4. The ultrasound diagnosis apparatus of claim 1, wherein the controller is configured to determine whether a movement of an object included in the region of interest exceeds a predetermined amount during a second time based on the ultrasound data,
wherein, when the movement of the object exceeds the predetermined amount, the controller is configured to additionally obtain the ultrasound data about the region of interest.

5. The ultrasound diagnosis apparatus of claim 1, further comprising a user input receiver configured to receive a user input,
wherein the controller is configured to receive the user input for storing the ultrasound still image and store the ultrasound still image corresponding to the user input.

6. The ultrasound diagnosis apparatus of claim 5, wherein the controller is configured to store the ultrasound still image to which an image quality parameter changed in a time interval corresponding to a time when the user input is received is applied.

7. The ultrasound diagnosis apparatus of claim 6, wherein the controller is configured to store the ultrasound still image obtained before the time interval.

8. The ultrasound diagnosis apparatus of claim 1, wherein the display is configured to display the ultrasound still image so that the ultrasound still image overlaps an outer portion of the real-time scan image.

9. The ultrasound diagnosis apparatus of claim 8, wherein the display is configured to sequentially display a plurality of the ultrasound still images to which image quality parameters that are changed are applied in an order in which the image quality parameters are applied.

10. The ultrasound diagnosis apparatus of claim 1, wherein the real-time scan image comprises a brightness (B) mode, a color (C) mode, a continuous wave Doppler (CW) mode, or a contrast enhanced image.

11. An ultrasound diagnosis method comprising:
obtaining a real-time scan image based on ultrasound data about a region of interest;
determining whether a movement of a probe occurs based on the ultrasound data;
when the movement of the probe does not occur for a first time or more, obtaining an ultrasound still image in which at least one image quality parameter according to a preset standard is changed based on the ultrasound data; and
displaying the real-time scan image and the ultrasound still image.

12. The ultrasound diagnosis method of claim 11, wherein the obtaining comprises, after the first time elapses, obtaining at least one ultrasound still image in which at least one image quality parameter is changed over time based on the ultrasound data.

13. The ultrasound diagnosis method of claim 11, wherein the obtaining comprises, after the first time elapses, obtaining at least one ultrasound still image in which at least one image quality parameter preset by a user is changed based on the ultrasound data.

14. The ultrasound diagnosis method of claim 11, further comprising:
determining whether a movement of an object included in the region of interest exceeds a predetermined amount during a second time based on the ultrasound data;
when the movement of the object exceeds the predetermined amount, additionally obtaining the ultrasound data about the region of interest.

15. The ultrasound diagnosis method of claim 11, further comprising:
receiving a user input for storing the ultrasound still image; and
storing the ultrasound still image corresponding to the user input.

16. The ultrasound diagnosis method of claim 15, wherein the storing comprises storing the ultrasound still image to which an image quality parameter changed in a time interval corresponding to a time when the user input is received is applied.

17. The ultrasound diagnosis method of claim 16, wherein the storing comprises storing the ultrasound still image obtained before the time interval.

18. The ultrasound diagnosis method of claim 11, wherein the displaying comprises displaying the ultrasound still image so that the ultrasound still image overlaps an outer portion of the real-time scan image.

19. The ultrasound diagnosis method of claim 18, wherein the displaying comprises sequentially displaying a plurality of the image quality parameters to which image quality parameters that are changed are applied in an order in which the image quality parameters are applied.

20. A computer-readable recording medium having embodied thereon a program for executing the ultrasound diagnosis method of claim 11.
